(19) United States
(12) Patent Application Publication
(10) Pub. No.: US 2019/0282641 A1
(43) Pub. Date: Sep. 19, 2019

(54) Generation of Tumor Endothelium Specific Viruses

(71) Applicant: BATU BIOLOGICS, INC., San Diego, CA (US)

(72) Inventors: Samuel C. Wagner, San Diego, CA (US); Thomas E. Ichim, San Diego, CA (US)

(21) Appl. No.: 16/356,803

(22) Filed: Mar. 18, 2019

Related U.S. Application Data
(60) Provisional application No. 62/644,207, filed on Mar. 16, 2018.

(51) Int. Cl. A61K 35/768; C12N 5/095; C12N 7/00; A61K 35/13; A61K 39/00

(52) U.S. Cl. CPC A61K 35/768; C12N 5/0695; C12N 7/00; A61K 35/13; A61K 39/0011; C12N 2710/24152; A61K 2039/585; C12N 2501/15; C12N 2501/02; C12N 2500/02; C12N 2710/24133; A61K 2039/5256; A61K 2039/575; C12N 2500/60

(57) ABSTRACT

Disclosed are means, methods, and compositions of matter useful for generation of viruses that selectively grow in the tumor endothelium and causes lysis or augmentation of tumor immunity. In one embodiment an oncolytic virus is selectively maintained in cells resembling tumor endothelium under conditions allowing for the oncolytic virus to capture immunogenic entities found on the cells resembling tumor endothelial cells. In another embodiment, the endothelial cells resembling tumor endothelial cells are utilized as a “Trojan horse” for selective delivery of virus into a tumor or tumor microenvironment.

### CROSS-REFERENCE TO RELATED APPLICATIONS

This Patent Application takes priority from Provisional Patent Application No. 62/644,207, titled Generation of Tumor Endothelium Specific Viruses, filed on Mar. 16, 2018, the contents of which are expressly incorporated herein by this reference as though set forth in their entirety and to which priority is claimed.

### BACKGROUND

Oncolytic viral therapy offers the possibility of not only killing tumor cells but also inducing a potent immune response. In fact, it is known that in many situations, virally induced immunity is associated with type 1 interferon signaling, which is also associated with suppression of cancer by immunotherapy. Thus, it may be conceptually possible that viral therapy of cancer potentially causes both immune activation as well as direct killing of the tumor.

Historically, the original oncolytic viral therapy for cancer to be approved by a regulatory agency was a genetically modified adenovirus named H101 that was manufactured by the Shanghai Sunway Biotech. This gained approval in 2005 from China's State Food and Drug Administration (SFDA) for the treatment of head and neck cancer. The mechanism of action of this study is that in normal cells, early proteins (E1A and E1B) are expressed when they are infected by adenovirus and early proteins are related with virus replication. The cell response for expression of E1A protein is to stimulate P53 expression. P53 is an important transcription regulation factor and a product of tumor suppressor gene. P53 overexpression results three changes in the cell: 1) cell arresting, 2) DNA damage-repair, 3) and inducing apoptosis. Those reactions are preventing replication of virus in normal cells. E1B-55KD can degrade P53 protein, which favoring virus replication. Comparing with wild virus, replication ability of E1B-55KD deleted virus was reduced; meanwhile, P53 couldn't be degraded effectively when E1B-55KD is deleted. Thus, oncorine® can't be replicated in normal cells. In P53 deficient cancer cells, as reactions of cells cannot be induced because of P53 deficiency, it is conducive for replication of reconstructed virus. The current view is that it is not only P53 mutation itself, but also P53 pathway deficiency, are conducive for selective replication of the oncolytic virus.

Another oncolytic adenovirus, named ONYX-015, is in ongoing clinical trials for the treatment of various solid tumors (in phase III for the treatment of recurrent head and neck cancer). Yet another example, oncolytic herpes simplex 1 (T-VEC) was genetically engineered to attenuate the virus virulence, increase selectivity for cancer cells and enhance antitumor immune response (through GM-CSF expression). Clinical efficacy in unresectable melanoma has been demonstrated in Phase II and Phase III clinical trials.

The majority of viral therapies for cancer are aimed at killing tumor cells. In the current invention we seek to overcome current limitations

### DESCRIPTION OF THE INVENTION

The invention teaches means of in vitro generation of viruses with immunogenic properties that induce cross-reactivity of immunity with cancer endothelial cells. The invention teaches use of endothelial cells made to resemble tumor endothelial cells, or tumor endothelial cells themselves to allow for viral growth, and subsequent uptake of antigens and incorporation of the antigens into the virus. Additionally, the invention teaches that growth of various oncolytic viruses can be achieved in endothelial cells and tumor endothelial cells in conditions of hypoxia. This allows the propagation of the virus in vitro and promotes antigenic uptake.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a polypeptide “comprises” an amino acid sequence when the amino acid sequence might be part of the final amino acid sequence of the polypeptide. Such a polypeptide can have up to several hundred additional amino acids residues.

“Consisting essentially of” means excluding other components or steps of any essential significance. Thus, a composition consisting essentially of the recited components would not exclude trace contaminants and pharmaceutically acceptable carriers. A polypeptide “consists essentially of” an amino acid sequence when such an amino acid sequence is present with eventually only a few additional amino acid residues.

“Consisting of” means excluding more than trace elements of other components or steps. For example, a polypeptide “consists of” an amino acid sequence when the polypeptide does not contain any amino acids but the recited amino acid sequence.

The terms “polypeptide”, “peptide” and “protein” refer to polymers of amino acid residues which comprise at least nine or more amino acids bonded via peptide bonds. The polymer can be linear, branched or cyclic and may comprise naturally occurring and/or amino acid analogs and it may be interrupted by non-amino acids. As a general indication, if the amino acid polymer is more than 50 amino acid residues, it is preferably referred to as a polypeptide or a protein whereas if it is 50 amino acids long or less, it is referred to as a “peptide”.

Within the context of the present invention, the terms “nucleic acid”, “nucleic acid molecule”, “polynucleotide” and “nucleotide sequence” are used interchangeably and define a polymer of any length of either polydeoxyribonucleotides (DNA) (e.g. cDNA, genomic DNA, plasmids, vectors, viral genomes, isolated DNA, probes, primers and any mixture thereof) or polyribonucleotides (RNA) (e.g. mRNA, antisense RNA, SiRNA) or mixed polyribopolydeoxyribonucleotides. They encompass single or double-stranded, linear or circular, natural or synthetic, modified or unmodified polynucleotides. Moreover, a polynucleotide may comprise non-naturally occurring nucleotides and may be interrupted by non-nucleotide components.

The term “analog” or “variant” as used herein refers to a molecule (polypeptide or nucleic acid) exhibiting one or more modification(s) with respect to the native counterpart. Any modification(s) can be envisaged, including substitution, insertion and/or deletion of one or more nucleotide/amino acid residue(s). Preferred are analogs that retain a degree of sequence identity of at least 80%, preferably at least 85%, more preferably at least 90%, and even more preferably at least 98% identity with the sequence of the native counterpart.

In a general manner, the term “identity” refers to an amino acid to amino acid or nucleotide to nucleotide correspondence between two polypeptide or nucleic acid sequences. The percentage of identity between two sequences is a function of the number of identical positions shared by the sequences, taking into account the number of gaps which need to be introduced for optimal alignment and the length of each gap. Various computer programs and mathematical algorithms are available in the art to determine the percentage of identity between amino acid sequences, such as for example the Blast program available at NCBI or ALIGN in Atlas of Protein Sequence and Structure (Dayhoffed, 1981, Suppl., 3: 482-9). Programs for determining identity between nucleotide sequences are also available in specialized data base (e.g. Genbank, the Wisconsin Sequence Analysis Package, BESTFIT, FASTA and GAP programs). For illustrative purposes, “at least 80% identity” means 80%, 81%, 82%, 83%, 84%, 85%, 86%, 87%, 88%, 89%, 90%, 91%, 92%, 93%, 94%, 95%, 96%, 97%, 98%, 99% or 100%.

As used herein, the term “isolated” refers to a protein, polypeptide, peptide, polynucleotide, vector, etc., that is removed from its natural environment (i.e. separated from at least one other component(s) with which it is naturally associated or found in nature). For example, a nucleotide sequence is isolated when it is separated of sequences normally associated with it in nature (e.g. dissociated from a genome) but it can be associated with heterologous sequences.

The term “obtained from”, “originating” or “originate” is used to identify the original source of a component (e.g. polypeptide, nucleic acid molecule) but is not meant to limit the method by which the component is made which can be, for example, by chemical synthesis or recombinant means.

As used herein, the term “host cell” should be understood broadly without any limitation concerning particular organization in tissue, organ, or isolated cells. Such cells may be of a unique type of cells or a group of different types of cells such as cultured cell lines, primary cells and dividing cells. In the context of the invention, the term “host cells” include prokaryotic cells, lower eukaryotic cells such as yeast, and other eukaryotic cells such as insect cells, plant and mammalian (e.g. human or non-human) cells as well as cells capable of producing the oncolytic virus and/or the immune checkpoint modulator(s) for use in the invention. This term also includes cells which can be or has been the recipient of the vectors described herein as well as progeny of such cells.

As used herein, the term “oncolytic virus” refers to a virus capable of selectively replicating in dividing cells (e.g. a proliferative cell such as a cancer cell) with the aim of slowing the growth and/or lysing the dividing cell, either in vitro or in vivo, while showing no or minimal replication in non-dividing cells. Typically, an oncolytic virus contains a viral genome packaged into a viral particle (or virion) and is infectious (i.e. capable of infecting and entering into a host cell or subject). As used herein, this term encompasses DNA or RNA vector (depending on the virus in question) as well as viral particles generated thereof.

The term “treatment” (and any form of treatment such as “treating”, “treat”) as used herein encompasses prophylaxis (e.g. preventive measure in a subject at risk of having the pathological condition to be treated) and/or therapy (e.g. in a subject diagnosed as having the pathological condition), eventually in association with conventional therapeutic modalities. The result of the treatment is to slow down, cure, ameliorate or control the progression of the targeted pathological condition. For example, a subject is successfully treated for a cancer if after administration of an oncolytic virus as described herein, the subject shows an observable improvement of its clinical status.

The term “administering” (or any form of administration such as “administered”) as used herein refers to the delivery to a subject of a therapeutic agent such as the oncolytic virus described herein.

As used herein, the term “proliferative disease” encompasses any disease or condition resulting from uncontrolled cell growth and spread including cancers as well as diseases associated to an increased osteoclast activity (e.g. rheumatoid arthritis, osteoporosis, etc) and cardiovascular diseases (restenosis that results from the proliferation of the smooth muscle cells of the blood vessel wall, etc). The term “cancer” may be used interchangeably with any of the terms “tumor”, “malignancy”, “neoplasm”, etc. These terms are meant to include any type of tissue, organ or cell, any stage of malignancy (e.g. from a prelesion to stage IV).

The term “subject” generally refers to an organism for whom any product and method of the invention is needed or may be beneficial. Typically, the organism is a mammal, particularly a mammal selected from the group consisting of domestic animals, farm animals, sport animals, and primates. Preferably, the subject is a human who has been diagnosed as having or at risk of having a proliferative disease such as a cancer. The terms “subject” and “patients” may be used interchangeably when referring to a human organism and encompasses male and female. The subject to be treated may be a newborn, an infant, a young adult or an adult.

The term “combination” or “association” as used herein refers to any arrangement possible of various components (e.g. an oncolytic virus and one or more substance effective in anticancer therapy). Such an arrangement includes mixture of the components as well as separate combinations for concomitant or sequential administrations. The present invention encompasses combinations comprising equal molar concentrations of each component as well as combinations with very different concentrations. It is appreciated that optimal concentration of each component of the combination can be determined by the artisan skilled in the art.

The term “immune checkpoint modulator” refers to a molecule capable of modulating the function of an immune checkpoint protein in a positive or negative way (in particular the interaction between an antigen presenting cell (APC) or a cancer cell and a T effector cell). The term “immune checkpoint” refers to a protein directly or indirectly involved in an immune pathway that under normal physiological conditions is crucial for preventing uncontrolled immune reactions and thus for the maintenance of self-tolerance and/or tissue protection. The one or more immune checkpoint modulator(s) in use herein may independently act at any step of the T cell-mediated immunity including clonal selection of antigen-specific cells, T cell activation, proliferation, trafficking to sites of antigen and inflammation, execution of direct effector function and signaling through cytokines and membrane ligands. Each of these steps is regulated by counterbalancing stimulatory and inhibitory signals that in fine tune the response. In the context of the present invention, the term encompasses immune checkpoint modulator(s) capable of down-regulating at least partially the function of an inhibitory immune checkpoint (antagonist) and/or immune checkpoint modulator(s) capable of up-regulating at least partially the function of a stimulatory immune checkpoint (agonist).

In one embodiment the invention teaches that ValloVax, a placental endothelium cell described in the following references, is capable of allowing for growth of oncolytic virus and promote not only trophism, but also stimulate immune response to tumor endothelium when administered in vivo. As substitution for ValloVax, other types of endothelial progenitor cells (EPC) may be used to stimulate immunity to tumor endothelium. The EPC, in one embodiment are a population of cells comprising cells having the surface marker CD44, cells having the surface marker CD13, cells having the surface marker CD90, cells having the surface marker CD105, cells having the surface marker ABCG2, cells having the surface marker HLA 1, cells having the surface marker CD34, cells having the surface marker CD133, cells having the surface marker CD117, cells having the surface marker CD135, cells having the surface marker CXCR4, cells having the surface marker c-met, cells having the surface marker CD31, cells having the surface marker CD14, cells having the surface marker Mac-1, cells having the surface marker CD11, cells having the surface marker c-kit cells having the surface marker SH-2, cells having the surface marker VE-Cadherin, VEGFR and cells having the surface marker Tie-2s. The EPC may be treated in a manner to mimic the tumor microenvironment, specifically, they may be grown under the acidic conditions in the tumor microenvironment and are incorporated by reference. In one embodiment of the invention, endothelial progenitor cells, or products thereof, are cultured under conditions in GCN2 kinase is activated, the conditions include culture in the presence of uncharged tRNA, tryptophan deprivation ,arginine deprivation, asparagine deprivation, and glutamine deprivation.

The invention teaches means of generating oncolytic viruses capable of stimulating immunity to tumor endothelium by culture such viruses in tumor endothelial cells, or cells generated to resemble tumor endothelial cells such as ValloVax placentally derived endothelial cells.

In one embodiment, the oncolytic virus is selected from the group consisting of reovirus, New Castle Disease virus (NDV), vesicular stomatitis virus (VSV), measles virus, influenza virus, Sinbis virus, adenovirus, poxvirus and herpes virus (HSV) and the like. In one embodiment, the oncolytic virus is a vaccinia virus. In a preferred embodiment, the vaccinia virus is engineered to lack thymidine kinase activity (e.g. the genome of the VV has an inactivating mutation in J2R gene to produce a defective TK phenotype). Alternatively, or in combination, the vaccinia virus is engineered to lack RR activity (e.g. the genome of the VV has an inactivating mutation in I4L and/or F4L gene to produce a defective RR phenotype). The oncolytic virus of the present invention can be obtained from any member of virus identified at present time provided that it is oncolytic by its propensity to selectivity replicate and kill dividing cells as compared to non-dividing cells. It may be a native virus that is naturally oncolytic or may be engineered by modifying one or more viral genes so as to increase tumor selectivity and/or preferential replication in dividing cells, such as those involved in DNA replication, nucleic acid metabolism, host tropism, surface attachment, virulence, lysis and spread One may also envisage placing one or more viral gene(s) under the control of event or tissue-specific regulatory elements (e.g. promoter).

In one embodiment, the oncolytic virus of the present invention is obtained from a herpes virus. The Herpesviridae are a large family of DNA viruses that all share a common structure and are composed of relatively large double-stranded, linear DNA genomes encoding 100-200 genes encapsided within an icosahedral capsid which is enveloped in a lipid bilayer membrane. Although the oncolytic herpes virus can be derived from different types of HSV, particularly preferred are HSV1 and HSV2. The herpes virus may be genetically modified so as to restrict viral replication in tumors or reduce its cytotoxicity in non-dividing cells. For example, any viral gene involved in nucleic acid metabolism may be inactivated, such as thymidine kinase (Martuza et al., 1991, Science 252: 854-6), ribonucleotide reductase (RR) (Boviatsis et al., Gene Ther. 1: 323-31; Mineta et al., 1994, Cancer Res. 54: 3363-66), or uracil-N-glycosylase (Pyles et al., 1994, J. Virol. 68: 4963-72). Another aspect involves viral mutants with defects in the function of genes encoding virulence factors such as the ICP34.5 gene (Chambers et al., 1995, Proc. Natl. Acad. Sci. USA 92: 1411-5). Representative examples of oncolytic herpes virus include NV1020 (e.g. Geevarghese et al., 2010, Hum. Gene Ther. 21(9): 1119-28) and T-VEC (Andtbacka et al., 2013, J. Clin. Oncol. 31, abstract number LBA9008).

In one embodiment, the oncolytic virus of the present invention is obtained from a morbillivirus which can be obtained from the paramyxoviridae family, with a specific preference for measles virus. Representative examples of oncolytic measles viruses include without limitation MV-Edm and HMWMAA. In another embodiment, the oncolytic virus of the present invention is obtained from an adenovirus. Methods are available in the art to engineer oncolytic adenoviruses. An advantageous strategy includes the replacement of viral promoters with tumor-selective promoters or modifications of the E1 adenoviral gene product(s) to inactivate its/their binding function with p53 or retinoblastoma (Rb) protein that are altered in tumor cells. In the natural context, the adenovirus E1B55 kDa gene cooperates with another adenoviral product to inactivate p53 (p53 is frequently dysregulated in cancer cells), thus preventing apoptosis. Representative examples of oncolytic adenovirus include ONYX-015.

In one embodiment, the oncolytic virus of the present invention is a poxvirus. As used herein the term “poxvirus” refers to a virus belonging to the Poxviridae family, with a specific preference for a poxvirus belonging to the Chordopoxviridae subfamily and more preferably to the Orthopoxvirus genus. Sequences of the genome of various poxviruses, for example, the vaccinia virus, cowpox virus, Canarypox virus, Ectromelia virus, Myxoma virus genomes. In one embodiment an oncolytic poxvirus that is used is an oncolytic vaccinia virus. Vaccinia viruses are members of the poxvirus family characterized by a 200 kb double-stranded DNA genome that encodes numerous viral enzymes and factors that enable the virus to replicate independently from the host cell machinery. The majority of vaccinia virus particles is intracellular (IMV for intracellular mature virion) with a single lipid envelop and remains in the cytosol of infected cells until lysis. The other infectious form is a double enveloped particle (EEV for extracellular enveloped virion) that buds out from the infected cell without lysing it.

It is known that several vaccinia virus strains exist. For use of the invention, Elstree, Wyeth, Copenhagen and Western Reserve strains are particularly preferred. The gene nomenclature used herein is that of Copenhagen vaccinia strain. It is also used herein for the homologous genes of other poxviridae unless otherwise indicated. However, gene nomenclature may be different according to the pox strain but correspondence between Copenhagen and other vaccinia strains are generally available in the literature. Preferably, the oncolytic vaccinia virus of the present invention is modified by altering for one or more viral gene(s). The modification(s) preferably lead(s) to the synthesis of a defective protein unable to ensure the activity of the protein produced under normal conditions by the unmodified gene (or lack of synthesis). Modifications encompass deletion, mutation and/or substitution of one or more nucleotide(s) (contiguous or not) within the viral gene or its regulatory elements. Modification(s) can be made in a number of ways known to those skilled in the art using conventional recombinant techniques. Exemplary modifications are disclosed in the literature with a specific preference for those altering viral genes involved in DNA metabolism, host virulence, IFN pathway.

In one embodiment of the invention, virus growth on endothelial cells is used to stimulate tumor immunity by means of antigens captured by the virus during passage in tumor endothelial cells. In other embodiments, combinations with other tumor inhibitor factors are used. In some embodiments, bacterial and viral infections have previously been demonstrated to possess ability to directly kill tumors through macrophage activation. This has been demonstrated for BCG, Corynebacterium parvum, streptococcal extracts such as OK-432, Mycobacterium smegmatis, Brucella abortus, Listeria monocytogenes, Leishmania braziliensis, Propionibacterium acnes, schistosoma mansoni, salmonella, nocardia rubra, E coli, candida albicans, Gordona aurantiaca, staphylococcus, mycoplasma, Newcastle Disease Virus, and bacteriophages.

In some embodiments embolization of tumor cells is used to generate an increased immune stimulatory environment. One of the other means of inducing “trauma” in the tumor is the utilization of localized ablation of blood vessels feeding the tumor while administration of localized chemotherapy. One example of such an approach is the surgical technique of trans arterial chemo embolization (TACE). In this procedure embolization is performed of the hepatic artery feeding the liver tumor, together with administration of lipiodol together. TACE is usually employed when standard therapy has failed or is known to be ineffective. TACE combines the advantages of intra-arterial chemotherapy, with the fact that embolization of the portal artery induces a preferential “starvation” of the tumor while sparing non-malignant hepatic tissue. Specifically, it is established that intra-arterial delivery of chemotherapy to the liver results in a tenfold higher intratumoral concentration as compared to administration through the portal vein. This is due in part to the observation that both primary and secondary liver tumors derive their blood supply preferentially from the hepatic artery. Anecdotal evidence suggested that embolization caused by thrombosis of the catheter during delivery of intraarterial chemotherapy as beneficial for inducing an improved tumor response. This prompted investigators to use surgical ablation or angiographic embolization to induce localized necrosis. Unfortunately, this approach, in absence of chemotherapy caused little effect on long-term survival. Therefore, the advantages of TACE is that both localized delivery of chemotherapy to the tumor occurs, while at the same time, the tumor blood flow is embolized, causing local tumor necrosis.

In some embodiments of the invention tumor antigens are incorporated into the virus in order to generate a dual anti-tumor and anti-tumor endothelial response. Antigens of interest include tumor antigens are selected from a group comprising of: ERG, WT1, ALS, BCR-ABL, Ras-mutant, MUC1, ETV6-AML, LMP2, p53 non-mutant, MYC-N, surviving, androgen receptor, RhoC, cyclin B1, EGFRvIII, EphA2, B cell or T cell idiotype, ML-IAP, BORIS, hTERT, PLAC1, HPV E6, HPV E7, OY-TES1, Her2/neu, PAX3, NY-BR-1, p53 mutant, MAGE A3, EpCAM, polysialic Acid, AFP, PAX5, NY-ESO1, sperm protein 17, GD3, Fucosyl GM1, mesothelin, PSMA, GD2, MAGE A1, sLe(x), HMWMAA, CYP1B1, sperm fibrous sheath protein, B7H3, TRP-2, AKAP-4, XAGE 1, CEA, Tn, GloboH, SSX2, RGS5, SART3, gp100, MelanA/MART1, Tyrosinase, GM3 ganglioside, Proteinase 3 (PR1), Page4, STn, Carbonic anhydrase IX, PSCA, Legumain, MAD-CT-1 (protamin2), PSA, Tie 2, MAD-CT2, PAP, PDGFR-beta, NA17, VEGFR2, FAP, LCK, Fos-related antigen, LCK, FAP.

In some embodiments, replication of tumor associated conditions is performed in vitro in order to allow for viral propagation and antigen uptake when viruses are growth on tumor endothelium or tumor endothelium-like cells. These conditions are described in numerous papers have which have characterized the acidic conditions in the tumor microenvironment and are incorporated by reference. Interestingly, tumor acidic conditions are believed to be associated with resistance to immunotherapy. In a recent study it was shown that an acidic pH environment blocked T-cell activation and limited glycolysis in vitro. IFNγ release blocked by acidic pH did not occur at the level of steady-state mRNA, implying that the effect of acidity was posttranslational. Acidification did not affect cytoplasmic pH, suggesting that signals transduced by external acidity were likely mediated by specific acid-sensing receptors, four of which are expressed by T cells. Notably, neutralizing tumor acidity with bicarbonate monotherapy impaired the growth of some cancer types in mice where it was associated with increased T-cell infiltration. Furthermore, combining bicarbonate therapy with anti-CTLA-4, anti-PD1, or adoptive T-cell transfer improved antitumor responses in multiple models, including cures in some subjects. In one embodiment of the invention, endothelial progenitor cells, or products thereof, are cultured under conditions in GCN2 kinase is activated, the conditions include culture in the presence of uncharged tRNA, tryptophan deprivation, arginine deprivation, asparagine deprivation, and glutamine deprivation.

In some embodiments, viruses are grown in extracellular matrix repopulated with cells that resemble cancer associated stromal cells, including fibroblasts, mesenchymal stem cells, endothelial progenitor cells, and combinations thereof. In order to generate an environment similar to the tumor microenvironment in terms of stiffness of fibrotic tissue, one approach involves crosslinking of ECM, the ECM includes collagen, fibrin, fibronectin and hyaluronic acid. In one specific embodiment, collagen is extracted from an extracellular matrix source, the sources may include decellularized tissues. In one embodiment ECM may be extracted from tumor tissues. In another embodiment, it may be extracted from placental sources. Means of extracting ECM from placental sources are known. For example, placenta may be manipulated to obtain the desired portion, e.g., to obtain a desired placental circulatory unit (e.g., a cotyledon) before the portion of the placenta is further processed (e.g., processed as described herein, e.g., decellularized). In certain embodiments, when only a portion of a placenta is used in the generation of the organoids described herein, the entire placenta is processed as desired (e.g., decellularized as described below), followed by isolation of the specific portion of the placenta to be used (e.g., by cutting or stamping out the desired portion of the placenta from the whole processed placenta). Once the placenta is prepared as above, and optionally perfused, it is decellularized in such a manner as to preserve the native structure of the placental vasculature, e.g., leave the placental vasculature substantially intact. As used herein, “substantially intact” means that the placental vasculature remaining after decellularization retains all, or most, of the gross structure of the placental vasculature prior to decellularization. In certain embodiments, the placental vasculature is capable of being re-seeded, e.g., with vascular endothelial cells or other cells, specifically tumor cell lines so as to recreate the tumor vasculature. The Placental tissue may be sterilized, e.g., by incubation in a sterile buffered nutrient solution containing antimicrobial agents, for example an antibacterial, an antifungal, and/or a sterilant compatible with the transplant tissue. The sterilized placental tissue may then be cryopreserved for further processing at a later time or may immediately be further processed according to the next steps of this process including a later cryopreservation of the tissue matrix or other tissue products of the process.

Means of decellularizing tissue including physical, chemical, and biochemical methods. See, e.g. U.S. Pat. No. 5,192,312 (Orton) which is incorporated herein by reference. Such methods may be employed in accordance with the process described herein. However, the decellularization technique employed preferably does not result in gross disruption of the anatomy of the placental tissue or substantially alter the biomechanical properties of its structural elements, and preferably leaves the placental vasculature substantially intact. In certain embodiments, the treatment of the placental tissue to produce a decellularized tissue matrix does not leave a cytotoxic environment that mitigates against subsequent repopulation of the matrix with cells that are allogeneic or autologous to the recipient. As used herein, cells and tissues that are “allogeneic” to the recipient are those that originate with or are derived from a donor of the same species as a recipient of the placental vascular scaffold, and “autologous” cells or tissues are those that originate with or are derived from a recipient of the placental vascular scaffold. In one embodiment the placental tissue is cryopreserved in the presence of one or more cryoprotectants. Colloid-forming materials may be added during freeze-thaw cycles to alter ice formation patterns in the tissue. For example, polyvinylpyrrolidone (10% w/v) and dialyzed hydroxyethyl starch (10% w/v) may be added to standard cryopreservation solutions (DMEM, 10% DMSO, 10% fetal bovine serum) to reduce extracellular ice formation while permitting formation of intracellular ice. In some embodiments, the placental tissue is decellularized using detergents or combinations thereof, for example, a nonionic detergent, e.g., Triton X-100, and an anionic detergent, e.g., sodium dodecyl sulfate, may disrupt cell membranes and aid in the removal of cellular debris from tissue. Preferably, residual detergent in the decellularized tissue matrix is removed, e.g., by washing with a buffer solution, so as to avoid interference with the later repopulating of the tissue matrix with viable cells.

1. A method of treating cancer comprising:
a) obtaining an oncolytic virus;
b) culturing the oncolytic virus in an environment so as to allow for the oncolytic virus to uptake antigens associated with the tumor endothelial cells; and
c) harvesting the oncolytic virus and administering the virus in a manner so as to stimulate immunity towards the tumor antigens which have been incorporated into the virus after culture of the virus in the tumor endothelial cells.

2. The method of claim 1, wherein the oncolytic virus is selected from the group consisting of: a) reovirus; b) Seneca Valley virus (SVV); c) vesicular stomatitis virus (VSV); d) Newcastle disease virus (NDV); e) herpes simplex virus (HSV); f) morbillivirus virus; g) retrovirus; h) influenza virus; i) Sinbis virus; j) poxvirus; k) vaccinia virus; and l) adenovirus.

3. The method of claim 2, wherein the vaccinia virus is altered in a manner to cause lack expression of thymidine kinase activity.

4. The method of claim 3, wherein the vaccinia virus is selected from a group of strains consisting of: a) Lister; b) Western Reserve (WR); c) Copenhagen (Cop); d) Bern; e) Paris; f) Tashkent; g) Tian Tan; h) Wyeth (DRYVAX); i) IHD-J; j) IHD-W; k) Brighton; l) Ankara; m) CVA382; n) Modified Vaccinia Ankara (MVA); o) Dairen I; p) LC16m8; q) LC16M0; r) LIVP; s) ACAM2000; t) WR 65-16; u) Connaught; v) New York City Board of Health (NYCBH); w) EM-63; and x) NYVAC strain.

5. The method of claim 1, wherein the culture allowing for the oncolytic virus to uptake antigens associated with tumor endothelial cells comprises culturing the virus on endothelial cells derived from placental endothelial progenitor cells.

6. The method of claim 5, wherein the placental endothelial progenitor cells are cultured under conditions of hypoxia, wherein the hypoxia constitutes growth of the endothelial progenitor cells under conditions of 0.01% oxygen to 5% oxygen

7. The method of claim 5, wherein the placental endothelial progenitor cells are cultured under conditions of acidosis, wherein the conditions of acidosis comprise culture of cells at a pH of 5.5 to 6.9.

8. The method of claim 5, wherein the placental endothelial progenitor cells are cultured in the presence of TGF-beta, wherein the TGF-beta concentration used for culture of the cells is between 0.01 nanograms per ml to 100 nanogram per ml.

9. The method of claim 5, wherein the placental endothelial progenitor cells are cultured in the presence of PGE-2, wherein the PGE-2 concentration used for culture of the cells is between 1 nanogram per ml to 1 microgram per ml.

10. The method of claim 1, wherein endothelial cells are treated with conditioned media of tumor cells in order to endow a tumor endothelial-like phenotype.

11. The method of claim 10, wherein the tumor endothelial-like phenotype consists of expression of markers selected from a group comprising of: a) FasL; b) FGF-receptor; c) VEGF-receptor; d) CD105; e) endosialin; and carbonic anhydrase.

12. The method of claim 1, wherein the tumor endothelial cells are generated by immortalization of endothelial cells derived from a tumor sample

13. The method of claim 1, wherein immunogenic genes are added to the oncolytic virus in order to promote immunogenicity.

14. The method of claim 13, wherein the immunogenic genes are cytokine genes, wherein the cytokine genes are selected from the group consisting of a) IL-2; b) IL-7; c) IL-8; d) IL-12; e) IL-15; f) IL-18; g) IL-21; h) TNF-alpha; i) IL-17; and j) IL-33.

15. The method of claim 1, wherein immunogenic genes are added to the oncolytic virus in order to promote immunogenicity are costimulatory molecule genes, wherein the costimulatory molecule genes are selected from a group comprising of: a) CD80; b) CD86; c) CD40; d) TIGIT; and e) ICOS ligand.

16. A method of treating cancer:
a) obtaining endothelial progenitor cells generated to resemble tumor endothelial cells;
b) infecting the endothelial progenitor cells with an oncolytic virus; and
c) administering the endothelial progenitor cells in patients suffering from cancer in a manner to allow for selective migration of the virally infected endothelial progenitor cells into the tumor vasculature.

17. The method of claim 16, wherein the endothelial cells resembling tumor vasculature are ValloVax cells.

18. The method of claim 16, wherein the endothelial progenitor cells are pretreated with hypoxia.

19. The method of claim 18, wherein the hypoxia treated is performed at a concentration and frequency to be sufficient to augment expression of CXCR-4.
